(19) United States
(12) Patent Application Publication
(10) Pub. No.: US 2002/0091107 A1
(43) Pub. Date: Jul. 11, 2002

(54) Oxazolidinone antibacterial agents

(76) Inventors: David J. Madar, Gurnee, IL (US); Daisy Pireh, Lincolnshire, IL (US); Hana Kopecka, Vernon Hills, IL (US); Stevan W. Djuric, Libertyville, IL (US); Paul E. Wiedeman, Deerfield, IL (US)

Correspondence Address: Steven F. Weinstock, Abbott Laboratories, Department 377 / AP6D-2, 100 Abbott Park Road, Abbott Park, IL, 60064-6050, US

(21) Appl. No.: 09/949,269

(22) Filed: Sep. 7, 2001

Related U.S. Application Data
(60) Provisional application No. 60/231,125, filed on Sep. 8, 2000.

(51) Int. Cl. C07D 413/02; C07D 417/02; A61K 31/655; A61K 31/422; A61K 31/427

(52) U.S. Cl. 514/151; 514/369; 514/376; 514/300; 546/113; 548/183; 548/229

(57) ABSTRACT

Compounds of formula (I)

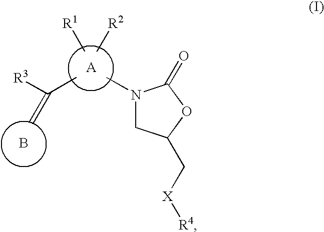

or therapeutically acceptable salts thereof, are useful for treating bacterial infections. Preparation of the compounds, compositions containing the compounds, and treatment of diseases using the compounds are disclosed.

This application claims priority to co-pending, U.S. Provisional Application Serial No. 60/231,125, filed Sep. 8, 2000.

### TECHNICAL FIELD

This invention is directed to compounds useful for treating bacterial infections, psoriasis, arthritis, and toxicity due to chemotherapy; preparation of the compounds; chemotherapeutic compositions comprising the compounds; and methods for treating diseases using the compounds.

### BACKGROUND OF THE INVENTION

Resistance to antibiotics once useful for treatment of bacterial infections resulting from pathogens such as Staphylococcus aureus, Staphylococcus epidermidis, and Enterococcus faecium has become a significant problem (Drugs Exp. Clin. Res. 1994, XX, 215-224; Am. J. Surg. 1995, 5A (Suppl.), 8S-12S; Drugs, 1994, 48, 678-688; and Current Pharmaceutical Design, 1996, Vol. 2, No. 2, pp 175-194). Thus, the development of new broad spectrum synthetic and semi-synthetic antibacterial compounds is the subject of constant current research.

One such class of compounds is the oxazolidinones, exemplified by eperezolid and linezolid, which constitute a class of orally active, synthetic antibacterial agents with good activity against Gram-positive bacteria (Current Pharmaceutical Design, op. cit.). Reference is also made to U.S. Pat. No. 4,977,173, European Patent 127,902-B1, and European Patent 316,594-B1, each of which teaches a series of antibacterial compounds comprising oxazolidinones connected to a substituted alkene through a phenyl ring. U.S. Pat. No. 6,040,306, the disclosure of which is hereinafter incorporated by reference into this specification, also teaches the use of oxazolidinones for treatment of psoriasis, arthritis, and toxicity due to chemotherapy.

Japanese Patent 7,173,159 also teaches a series of substituted oxazolidinones, although they are used as blood sugar-reducing agents.

Given these and other reports on the therapeutic benefit of oxazolidinone antibacterials, the loss of activity among antibacterials which were once efficacious for treatment of certain Gram-positive bacteria, and the continuing need for treatment of diseases such as psoriasis, arthritis, and toxicity due to chemotherapy, there is a continuing need for the development of novel oxazolidinone drugs with modified or improved profiles of activity.

### SUMMARY OF THE INVENTION

In its principle embodiment, the present invention provides an antibacterial agent of formula (I)

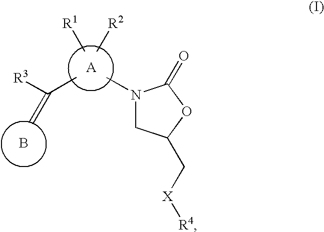

or a therapeutically acceptable salt thereof, wherein

A is selected from phenyl and a five- or six-membered aromatic ring containing one to three atoms selected from N, O, and S, and the remaining atoms are carbon, wherein A is substituted through carbon atoms in the ring;

B is heterocycle;

X is selected from the group consisting of O, S, S(O), SO₂, and NR⁵;

R¹ and R² are independently selected from the group consisting of hydrogen, alkoxy, alkyl, amino, cycloalkyl, halo, haloalkyl, hydroxy, and perfluoroalkyl;

R³ is selected from the group consisting of hydrogen, alkoxy, alkyl, amino, aryl, cyano, halo, haloalkoxy, hydroxy, and nitro;

R⁴ is selected from the group consisting of alkanoyl, alkoxycarbonyl, amido, aryl, aryloyl, heteroaryl, and heteroaryloyl; and

R⁵ is selected from the group consisting of hydrogen, alkyl, and arylalkyl;

with the proviso that when B is 2,4-dioxo-1,3-thiazolidin-5-yl and X is O, R⁴ is other than phenyl.

### DETAILED DESCRIPTION OF THE INVENTION

The compounds of the present invention are substituted oxazolidinones which are useful for the treatment of bacterial infections. In its principle embodiment, the present invention provides compounds of formula (I)

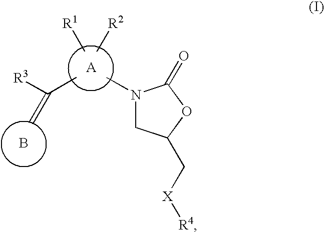

or therapeutically acceptable salts thereof, wherein A, B, X, R¹, R²,R³, and R⁴ are as previously defined.

The compounds of the invention comprise oxazolidinones connected to a substituted alkene through ring A. Ring A is a stable, aromatic group substituted through carbon atoms in the ring. Preferably, ring A is phenyl, although heteroaryl rings such as furanyl, thienyl, pyrrolyl, oxazolyl, thiazolyl, imidazolyl, pyrazolyl, isoxazolyl, isothiazolyl, pyridyl, pyridazinyl, pyrimidinyl, and pyrazinyl are within the scope of the invention. Ring A can be further substituted by independent replacement of one or two hydrogen atoms thereon by substituents defined by R¹ and R² so that, for instance and by way of example only, ring A can be substituted by halo, preferably fluoro. Lines drawn into ring A (such as from R¹ and R²) indicate that the bonds can be attached to any substitutable ring carbon atom.

R³ is preferably hydrogen, although groups such as alkyl, amino, cyano, halo, and nitro are within the scope of the invention.

Ring B is a stable, non-aromatic mono- or bicyclic optionally substituted heterocycle group attached to the parent molecular moiety through a substitutable carbon atom in the ring. Preferably, ring B is dihydropyrazole, imidazolidine, indoline, pyrrolidine, or thiazolidine, although heterocycle groups such as dihydrobenzofuran, dihydrobenzimidazole, dihydrothiazole, dihydrobenzothiophene, octahydrobenzofuran, octahydroindole, oxathiolane, oxazolidine, and the like, are within the scope of the invention.

A preferred embodiment of compounds of formula (I) are compounds of formula (II)

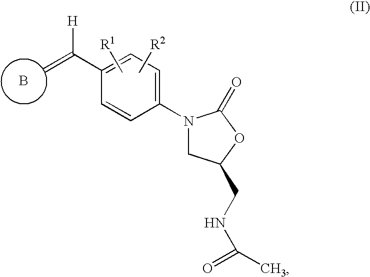

or therapeutically acceptable salts thereof, wherein B, R¹, and R² are as previously defined.

In another preferred embodiment are compounds of formula (II)

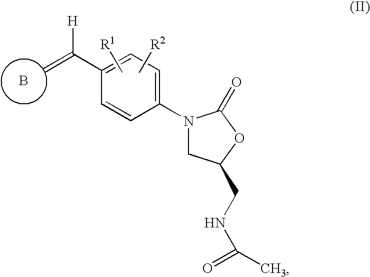

or therapeutically acceptable salts thereof, wherein B is heterocycle wherein the heterocycle is selected from the group consisting of

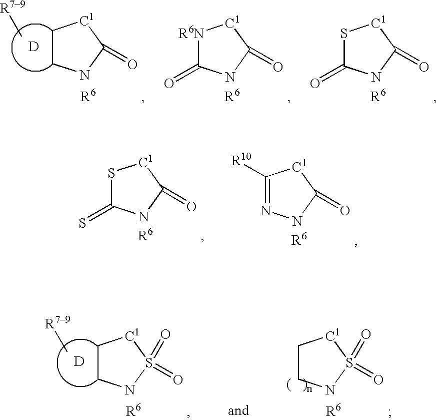

wherein C¹ s the point of attachment to the parent molecular moiety;

n is 1 or 2;

D is selected from phenyl and a five- or six-membered aromatic ring containing one or two atoms selected from N, O, and S, and the remaining atoms are carbon, wherein the N is optionally oxidized, and wherein D is fused through carbon atoms in the ring;

R¹ and R² are independently selected from the group consisting of alkoxy, alkyl, and halo;

each R⁶ is independently selected from the group consisting of hydrogen, alkanoyl, alkoxycarbonyl, alkyl, amido, aminoalkyl, aminosulfonyl, aryl, heteroaryl, hydroxyalkyl, and a nitrogen protecting group; and

R⁷-R¹⁰ are independently selected from the group consisting of hydrogen, alkoxy, alkoxycarbonyl, alkyl, amido, amino, aminosulfonyl, azido, carboxy, cyano, halo, haloalkoxy, haloalkyl, mercapto, nitro, perfluoroalkoxy, perfluoroalkyl, and thioalkoxy.

In still another preferred embodiment are compounds of formula (I) wherein A is phenyl.

In a particularly preferred embodiment are compounds of formula (II) wherein B is

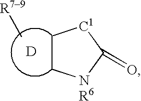

specific examples of which include the compounds

N-(((5S)-3-(3-fluoro-4-((E)-(2-oxo-1,2-dihydro-3H-pyrrolo(2,3-b)pyridin-3-ylidene)methyl)phenyl)-2-oxo-1,3-oxazolidin-5-yl)methyl)acetamide,

N-(((5S)-3-(4-((Z)-(4,5-dimethyl-2-oxo-1,2-dihydro-3H-indol-3-ylidene)methyl)phenyl)-2-oxo-1,3-oxazolidin-5-yl)methyl)acetamide,

N-(((5S)-2-oxo-3-(4-((E)-(2-oxo-1,2-dihydro-3H-indol-3-ylidene)methyl)phenyl)-1,3-oxazolidin-5-yl)methyl)acetamide,

N-(((5S)-3-(4-((Z)-(5-chloro-2-oxo-1 ,2-dihydro-3H-indol-3-ylidene)methyl)phenyl)-2-oxo-1,3-oxazolidin-5-yl)methyl)acetamide,

N-(((5S)-3-(4-((E)-(5-chloro-2-oxo-1,2-dihydro-3H-indol-3-ylidene)methyl)phenyl)-2-oxo-1,3-oxazolidin-5-yl)methyl)acetamide,

N-(((5S)-3-(4-((E)-(5-nitro-2-oxo-1,2-dihydro-3H-indol-3-ylidene)methyl)phenyl)-2-oxo-1,3-oxazolidin-5-yl)methyl)acetamide,

N-(((5S)-3-(3-fluoro-4-((E)-(2-oxo-1,2-dihydro-3H-indol-3-ylidene)methyl)phenyl)-2-oxo-1,3-oxazolidin-5-yl)methyl)acetamide,

N-(((5S)-3-(3-fluoro-4-((Z)-(1-methyl-2-oxo-1,2-dihydro-3H-indol-3-ylidene)methyl)phenyl)-2-oxo-1,3-oxazolidin-5-yl)methyl)acetamide,

N-(((5S)-3-(3-fluoro-4-((E)-(1-methyl-5-nitro-2-oxo-1,2-dihydro-3H-indol-3-ylidene)methyl)phenyl)-2-oxo-1,3-oxazolidin-5 -yl)methyl)acetamide,

N-(((5 S)-3-(4-((E)-(6-chloro-2-oxo-1,2-dihydro-3H-indol-3-ylidene)methyl)-3-fluorophenyl)-2-oxo-1,3-oxazolidin-5-yl)methyl)acetamide,

N-(((5S)-3-(4-((E)-(5-methoxy-2-oxo-1,2-dihydro-3H-indol-3-ylidene)methyl)-3-fluorophenyl)-2-oxo-1,3-oxazolidin-5-yl)methyl)acetamide,

N-(((5S)-3-(4-((Z)-(5-chloro-2-oxo-1,2-dihydro-3H-indol-3-ylidene)methyl)-3-fluorophenyl)-2-oxo-1,3-oxazolidin-5-yl)methyl)acetamide,

N-(((5S)-3-(4-((E)-(1-methyl-2-oxo-1,2-dihydro-3H-indol-3-ylidene)methyl)phenyl)-2-oxo-1,3-oxazolidin-5-yl)methyl)acetamide,

N-[((5S)-3-{4-[(E)-(6-chloro-2-oxo-1,2-dihydro-3H-indol-3-ylidene)methyl]phenyl}-2-oxo-1,3-oxazolidin-5-yl)methyl]acetamide,

N-[((5S)-3-{4-[(E)-(5,6-dimethoxy-2-oxo-1,2-dihydro-3H-indol-3-ylidene)methyl]-3-fluorophenyl}-2-oxo-1,3-oxazolidin-5-yl)methyl]acetamide,

N-[((5S)-3-{4-[(Z)-(5-methoxy-2-oxo-1,2-dihydro-3H-indol-3-ylidene)methyl]phenyl}-2-oxo-1,3-oxazolidin-5-yl)methyl]acetamide,

N-[((5S)-3-{4-[(E)-(1-acetyl-2-oxo-1,2-dihydro-3H-indol-3-ylidene)methyl]-3-fluorophenyl}-2-oxo-1,3-oxazolidin-5-yl)methyl]acetamide,

N-[((5S)-3-{4-[(Z)-(4,7-dimethyl-2-oxo-1,2-dihydro-3H-indol-3-ylidene)methyl]-3-fluorophenyl}-2-oxo-1,3-oxazolidin-5-yl)methyl]acetamide,

N-{[(5S)-3-(3-fluoro-4-{(E)-[2-oxo-5-(trifluoromethoxy)-1,2-dihydro-3H-indol-3-ylidene]methyl}phenyl)-2-oxo-1,3-oxazolidin-5-yl]methyl}acetamide, and

N-{[(5S)-3-(3-fluoro-4-{(Z)-[2-oxo-5-(trifluoromethoxy)-1,2-dihydro-3H-indol-3-ylidene]methyl}phenyl)-2-oxo-1,3 -oxazolidin-5-yl]methyl}acetamide.

In srtill another preferred embodiment are compounds of formula (II) wherein B is

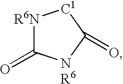

a specific example of which includes

N-(((5S)-3-(4-((E)-(3-methyl-2,5-dioxo-4-imidazolidinylidene)methyl)phenyl)-2-oxo-1,3-oxazolidin-5-yl)methyl)acetamide.

In still another preferred embodiment are compounds of formula (II) wherein B is

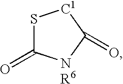

specific examples of which include the compounds

N-(((5S)-3-(4-((Z)-(2,4-dioxo-1,3-thiazolidin-5-ylidene)methyl)phenyl)-2-oxo-1,3-oxazolidin-5-yl)methyl)acetamide and

N-(((5S)-3-(4-((Z)-(2,4-dioxo-1,3-thiazolidin-5-ylidene)methyl)-3-fluorophenyl)-2-oxo-1,3-oxazolidin-5-yl)methyl)acetamide.

In still another preferred embodiment are compounds of formula (II) wherein B is

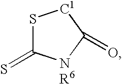

a specific example of which is the compound

N-[((5S)-2-oxo-3-{4-[(Z)-(4-oxo-2-thioxo-1,3-thiazolidin-5-ylidene)methyl]phenyl}-1,3-oxazolidin-5-yl)methyl]acetamide.

In still another preferred embodiment are compounds of formula (II) wherein B is

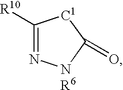

specific examples of which are the compounds

N-(((5S)-3-(4-((Z)-(3-tert-butyl-1-methyl-5-oxo-1,5-dihydro-4H-pyrazol-4-ylidene)methyl)phenyl)-2-oxo-1,3-oxazolidin-5-yl)methyl)acetamide and

N-(((5S)-3-(4-((Z)-(3-tert-butyl-5-oxo-1-phenyl-1,5-dihydro-4H-pyrazol-4-ylidene)methyl)phenyl)-2-oxo-1,3-oxazolidin-5-yl)methyl)acetamide.

In still another embodiment, the present invention provides a pharmaceutical composition comprising a compound of formula (I), or a therapeutically acceptable salt thereof, in combination with a therapeutically acceptable carrier.

In still another embodiment, the present invention provides a method for treating bacterial infections in a patient in recognized need of such treatment comprising administering to the patient a therapeutically acceptable amount of a compound of formula (I), or a therapeutically acceptable salt thereof.

In still another embodiment, the present invention provides a method for treating psoriasis in a patient comprising administering to the patient a therapeutically acceptable amount of a compound of formula (I), or a therapeutically acceptable salt thereof.

In still another embodiment, the present invention provides a method for treating arthritis in a patient comprising administering to the patient a therapeutically acceptable amount of a compound of formula (I), or a therapeutically acceptable salt thereof.

In still another embodiment, the present invention provides a method for treating toxicity due to chemotherapy in a patient comprising administering to the patient a therapeutically acceptable amount of a compound of formula (I), or a therapeutically acceptable salt thereof.

In still another embodiment, the present invention provides a composition comprising a compound of formula (II), or a therapeutically acceptable salt thereof and a therapeutically acceptable carrier.

In still another embodiment, the present invention provides a method for treating bacterial infections in a patient in recognized need of such treatment comprising administering to the patient a therapeutically acceptable amount of a compound of formula (II), or a therapeutically acceptable salt thereof.

In still another embodiment, the present invention provides a method for treating psoriasis in a patient comprising administering to the patient a therapeutically acceptable amount of a compound of formula (II), or a therapeutically acceptable salt thereof.

In still another embodiment, the present invention provides a method for treating arthritis in a patient comprising administering to the patient a therapeutically acceptable amount of a compound of formula (II), or a therapeutically acceptable salt thereof.

In still another embodiment, the present invention provides a method for treating toxicity due to chemotherapy in a patient comprising administering to the patient a therapeutically acceptable amount of a compound of formula (II), or a therapeutically acceptable salt thereof.

Because asymmetric centers exist in the present compounds, the invention contemplates stereoisomers and thereof. Individual stereoisomers of compounds are prepared by synthesis from starting materials containing the chiral centers or by preparation of mixtures of enantiomeric products followed by separation such as conversion to a mixture of diastereomers followed by separation or recrystallization, chromatographic techniques, or direct separation of the enantiomers on chiral chromatographic columns. Starting compounds of particular stereochemistry are either commercially available or are made by the methods described below and resolved by techniques well known in the art.

The present compounds may exhibit the phenomena of tautomerism or structural isomerism. For example, the compounds described herein may adopt an E or Z conformation about the double bond connecting the A ring to the B ring, or may be mixtures of E and Z isomers. As the drawings within this specification can only represent one possible tautomeric or structural isomeric form, it should be understood that the invention encompasses any tautomeric or structural isomeric form, or mixtures thereof, which possess the ability to inhibit bacterial growth, and is not limited to any one tautomeric or structural isomeric form utilized within the drawings. As used herein, the term “E” represents higher order substituents on opposite sides of a carbon-carbon double bond, and the term “Z” represents higher order substituents on the same side of a carbon-carbon double bond.

In addition to the compounds of the present invention and their pharmaceutically acceptable salts, the invention is further directed, where applicable, to unsolvated as well as solvated forms of the compounds (e.g. hydrated forms) having the ability to inhibit bacterial growth.

As used in the present specification the following terms have the meanings indicated:

The term “additive,” as used herein, means a catalyst or reagent which advances the course of the reaction. Examples of additives include H₃PO₄, pyridinium trifluoroacetate, silica gel, TEA, pyridine, and quinoline. The term “additive,” also means dehydrating agents such as magnesium sulfate, silica gel, and molecular sieves. The term “additive,” also means monodentate phosphorus-containing ligands of formulas P(R^{c})₃ (phosphines), P(OR^{d})₃ (phosphites) and As(R^{c})₃ (arsines), wherein each R^{c} is independently hydrogen; alkyl such as methyl, ethyl, and tert-butyl; cycloalkyl such as cyclopropyl and cyclohexyl; optionally substituted aryl such as phenyl, naphthyl, and ortho-tolyl; and optionally substiuted heteroaryl such as furyl and pyridyl; and wherein each R^{d} is independently alkyl such as methyl, ethyl, and tert-butyl; cycloalkyl such as cyclopropyl and cyclohexyl; optionally substituted aryl such as phenyl, naphthyl, and ortho-tolyl; and optionally substituted heteroaryl such as furyl and pyridyl. Specific examples of these additives include tri(alkyl)phosphines such as trimethylphosphine, triethylphosphine, tributylphosphine, and the like; tri(cycloalkyl)phosphines such as tricyclopropylphosphine, tricyclohexylphosphine, and the like; tri(aryl)phosphines such as triphenylphosphine, trinaphthylphosphine, and the like; tri(heteroaryl)phosphines such as tri(fur-2-yl)phosphine, tri(pyrid-3-yl)phosphine, and the like; tri(alkyl)phosphites such as trimethylphosphite, triethylphosphite, tributylphosphite, and the like; tri(cycloalkyl)-phosphites such as tricyclopropylphosphite, tricyclohexylphosphite, and the like; tri(aryl)phosphites such as triphenylphosphite, trinaphthylphosphite, and the like; tri(heteroaryl)phosphites such as tri(fur-2-yl)phosphite, tri(pyrid-3-yl)phosphite, and the like; and triphenylarsine, and the like. The term “additive,” also means bidentate phosphines such as 1,4-bis(diphenylphosphino)butane (DPPB), 1,2-bis(diphenyl-phosphino)ethane (DPPE), 1,1-bis(diphenylphosphino)methane (DPPM), 1,2-bis(dimethyl-phosphino)ethane (DMPE), 1,1′-bis(diphenylphosphino)ferrocene (DPPF), and the like. The term “additive,” also means copper salts such as copper(I) iodide and copper(I) chloride. It should be understood that multiple additives may be added to a reaction to promote the progress thereof. As an example, a transition metal coupling reaction can employ a copper salt, such as copper(I) iodide, and also a bidentate phospine ligand, such as DPPE.

The term “alkanoyl,” as used herein, represents an alkyl group attached to the parent molecular moiety through a carbonyl group.

The term “alkoxy,” as used herein, represents an alkyl group attached to the parent molecular moiety through an oxygen atom.

The term “alkoxycarbonyl,” as used herein, represents an alkoxy group attached to the parent molecular moiety through a carbonyl group.

The term “alkyl,” as used herein, represents a monovalent group derived from a straight or branched chain saturated hydrocarbon by the removal of a single hydrogen atom.

The term “amido,” as used herein, represents an amino group attached to the parent molecular moiety through a carbonyl group.

The term “amino,” as used herein, represents —NR¹¹R¹², wherein R¹¹ and R¹² are independently selected from the group consisting of hydrogen, alkanoyl, alkyl, cycloalkyl, (cycloalkyl)alkyl, a nitrogen protecting group, and phenyl; or R¹¹ and R ¹², together with the nitrogen atom to which they are attached, form a ring selected from the group consisting of morpholinyl, piperazinyl, piperidinyl, pyrrolidinyl, thiomorpholinyl, and thiomorpholinyl dioxide.

The term “aminoalkyl,” as used herein, represents an amino group attached to the parent molecular moiety through an alkyl group.

The term “aminosulfonyl,” as used herein, represents an amino group attached to the parent molecular moiety through a sulfonyl group.

The term “aryl,” as used herein, represents dihydronaphthyl, fluorenyl, indanyl, indenyl, naphthyl, phenyl, and tetrahydronaphthyl. Aryl groups having an unsaturated or partially saturated ring fused to an aromatic ring can be attached through the saturated or the unsaturated part of the group. The aryl groups of this invention can be optionally substituted with one, two, three, four, or five substituents independently selected from the group consisting of alkoxy, alkoxycarbonyl, alkyl, amido, amino, aminosulfonyl, azido, carboxy, cyano, halo, haloalkoxy, haloalkyl, mercapto, nitro, perfluoroalkyl, and thioalkoxy.

The term “arylalkyl,” as used herein, represents an aryl group attached to the parent molecular moiety through an alkyl group. Representative arylalkyl groups are benzyl, 4-methoxybenzyl, 2,4-dimethoxybenzyl, and the like.

The term “aryloyl,” as used herein, represents an aryl group attached to the parent molecular moiety through a carbonyl group.

The term “azido,” as used herein, represents —N₃.

The term “carbonyl,” as used herein, represents —C(O)—.

The term “carboxy,” as used herein, represents —CO₂H.

The term “cyano,” as used herein, represents —CN.

The term “cycloalkyl,” as used herein, represents a saturated cyclic, bicyclic, or tricyclic hydrocarbon ring system having three to twelve carbon atoms. Examples of cycloalkyl groups include cyclopropyl, cyclopentyl, bicyclo[3.1.1]heptyl, adamantyl, and the like. The cycloalkyl groups of this invention can be optionally substituted with one, two, three, or four substituents independently selected from the group consisting of alkoxy, alkoxycarbonyl, alkyl, azido, carboxy, cyano, halo, haloalkoxy, haloalkyl, nitro, and thioalkoxy.

The term “(cycloalkyl)alkyl,” as used herein, represents a cycloalkyl group attached to the parent molecular moiety through an alkyl group.

The term “halo,” as used herein, represents F, Cl, Br, or I.

The term “haloalkoxy,” as used herein, represents a haloalkyl group attached to the parent molecular moiety through an oxygen atom.

The term “haloalkyl,” as used herein, represents an alkyl group substituted by one, two, three, or four halogen atoms.

The term “heteroaryl,” as used herein, represents a cyclic aromatic group having five or six ring atoms wherein at least one ring atom is selected from the group consisting of oxygen, sulfur, and nitrogen, and the remaining ring atoms are carbon. The five-membered rings have two double bonds, and the six-membered rings have three double bonds. The term “heteroaryl” also includes bicyclic groups in which the heteroaryl ring is fused to an aryl group. Examples of heteroaryl groups include furyl, imidazolyl, thienyl, pyridinyl, pyrimidinyl, indolyl, benzothiazolyl, benzoxazolyl, benzimidazolyl, and quinolinyl. The heteroaryl groups of the present invention are connected to the parent molecular moiety through a carbon atom in the ring or, as exemplified by imidazolyl, indolyl, and benzimidazolyl, through either a carbon atom or nitrogen atom in the ring. The heteroaryl groups of this invention can be optionally substituted with one, two, three, four, or five substituents independently selected from the group consisting of alkoxy, alkoxycarbonyl, alkyl, amido, amino, aminosulfonyl, azido, carboxy, cyano, halo, haloalkoxy, haloalkyl, mercapto, nitro, perfluoroalkyl, and thioalkoxy.

The term “heteroaryloyl,” as used herein, represents a heteroaryl group attached to the parent molecular moiety through a carbonyl group.

The term “heterocycle,” as used herein, represents a non-aromatic five- or six-membered ring having two or three heteroatoms independently selected from oxygen, sulfur, and nitrogen, wherein the nitrogen and the sulfur are optionally oxidized. The five-membered ring has zero to one double bond and the six-membered ring has zero to two double bonds. The heterocycle groups of the present invention are connected to the parent molecular moiety through a substitutable carbon atom in the ring. Heterocycle groups can be optionally fused to a ring selected from the group consisting of aryl, heteroaryl, heterocycle, and cycloalkyl to provide a bicyclic group which is attached to the parent molecular moiety through a substitutable carbon atom on the heterocycle part of the group. The term “heterocycle” also represents a non-aromatic five-or six-membered ring having one heteroatom selected from the group consisting of oxygen, sulfur, and nitrogen, wherein the ring is fused to a second ring selected from the group consisting of aryl, heteroaryl, heterocycle, and cycloalkyl to provide a bicyclic group, which is attached to the parent molecular moiety through a carbon atom on the heterocycle part of the group. The heterocycle groups of this invention can be optionally substituted with one, two, three, or four substituents independently selected from the group consisting of alkoxy, alkoxycarbonyl, alkyl, amido, amino, aminoalkyl, aminosulfonyl, aryl, azido, carboxy, cyano, cycloalkyl, halo, haloalkoxy, haloalkyl, heteroaryl, hydroxy, hydroxyalkyl, mercapto, nitro, a nitrogen protecting group, oxo, perfluoroalkyl, thioalkoxy, and (thio)oxo. Examples of heterocyclic groups of the present invention include, but are not limited to, dihydropyrazole, imidazolidine, indoline, pyrrolidine, thiazolidine, dihydrobenzofuran, dihydrobenzimidazole, dihydrothiazole, dihydrobenzothiophene, octahydrobenzofuran, octahydroindole, oxathiolane, oxazolidine, and the like.

The term “hydroxy,” as used herein, represents —OH.

The term “hydroxyalkyl,” as used herein, represents a hydroxy group attached to the parent molecular moiety through an alkyl group.

The term “mercapto,” as used herein, represents —SH.

The term “nitro,” as used herein, represents —NO₂.

The term “nitrogen protecting group,” as used herein, represents groups intended to protect an amino group against undesirable reactions during synthetic procedures. Common N-protecting groups comprise acyl groups such as acetyl, benzoyl, 2-bromoacetyl, 4-bromobenzoyl, tert-butylacetyl, carboxaldehyde, 2-chloroacetyl, 4-chlorobenzoyl, a-chlorobutyryl, 4-nitrobenzoyl, o-nitrophenoxyacetyl, phthalyl, pivaloyl, propionyl, trichloroacetyl, and trifluoroacetyl; sulfonyl groups such as benzenesulfonyl, and p-toluenesulfonyl; carbamate forming groups such as benzyloxycarbonyl, benzyloxycarbonyl (Cbz), tert-butyloxycarbonyl (Boc), p-chlorobenzyloxycarbonyl, p-methoxybenzyloxycarbonyl, and the like.

The term “oxo,” as used herein, represents ═O.

The term “perfluoroalkoxy,” as used herein, represents an perfluoroalkyl group attached to the parent molecular moiety through an oxygen atom.

The term “perfluoroalkyl,” as used herein, represents an alkyl group wherein each hydrogen radical bound to the alkyl group has been replaced by a fluoride radical.

The term “sulfonyl,” as used herein, represents —SO₂—.

The term “thioalkoxy,” as used herein, represents an alkyl group attached to the parent molecular moiety through a sulfur atom.

The term “(thio)oxo,” as used herein, represents ═S.

The compounds of the present invention can exist as therapeutically acceptable salts. The term “therapeutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The present compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to parent compounds of formula (I) for example, by hydrolysis in blood.

In accordance with methods of treatment and pharmaceutical compositions of the invention, the compounds can be administered alone or in combination with other antibacterial agents. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The antibacterial effect of parenterally administered compounds can be prolonged by slowing their absorption. One way to slow the absorption of a particular compound is administering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefor.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Determination of Biological Activity

The minimum inhibitory concentrations (MIC's) of the compounds for the microorganisms listed in Table 1 were determined by the procedure described in National Committee for Clinical Laboratory Standards. 2000. Methods for Dilution Antimicrobial Susceptibility Tests for Bacteria That Grow Aerobically, 5th ed. Approved Standard: M7-A5 (NCCLS, Wayne, Pa.). Briefly, the compounds were dissolved in DMSO to 2 mg/mL and diluted in the appropriate susceptibility test medium to a concentration of 256 μg/mL. Serial two-fold dilutions were made in microtiter plates to achieve a final volume of 50 μL. Inocula for each organism were prepared by making a standard suspension in sterile saline with turbidity equivalent to that of a 0.5 McFarland Standard from an 18 to 24 hour culture grown on agar plates at 35° C. The standard suspension of each organism was diluted 100-fold in the appropriate medium and further diluted 2-fold by adding 50 μL to the medium containing antibiotic to achieve a final density of 5×10⁵ CFU/mL. Microdilution plates were incubated for 16 to 20 hours at 35° C. in ambient air. Each plate was visually inspected, and MIC's were recorded as the lowest concentration of drug which yielded no growth, a slight haze, or sparsely isolated colonies on the inoculum spot as compared to the growth control.

The compounds inhibited the growth of these bacteria with MIC's in a range of about 2 μg/mL to about 128 μ/mL; in a more preferred range, the compounds inhibited the growth of bacteria with MIC's in a range of about 2 μg/mL to about 16 μg/mL; and in a most preferred range, the compounds inhibited the growth of bacteria with MIC's in a range of about 2 μg/mL to about 8 μg/mL.

Thus, the compounds are useful for treating bacterial infections including, but not limited to, those shown in Table 1.

TABLE 1

Microorganism
Staphylococcus aureus
NCTC 10649M
Staphylococcus epidermidis
3519
Moraxella catarrhalis
2604
Enterococcus faecium
ATCC GYR 1632
Streptococcus pneumoniae
ATCC 6303

Synthetic Methods

Abbreviations which have been used in the descriptions of the scheme and the examples that follow are: dba for dibenzylideneacetone; BINAP for 2,2′-bis(diphenylphosphino)-1,1′-binaphthyl; dba for dibenzylideneacetone, DPPE for 1,2-bis(diphenylphosphino)ethane, DPPF for 1,1′-bis(diphenylphosphino)ferrocene; THF for tetrahydrofuran; and TFP for tris-2-furylphosphine.

The compounds and processes of the present invention will be better understood in connection with the following synthetic schemes which illustrate the methods by which the compounds of the invention may be prepared. It will be readily apparent to one of ordinary skill in the art that the compounds can be synthesized by substitution of the appropriate reactants and reagents in these syntheses, and that the steps themselves can be conducted in varying order. It will also be apparent that protection and deprotection steps can be performed to successfully complete the syntheses of the compounds. It should be understood that multiple additives may be added to a reaction to promote the progress thereof. As an example, a transition metal coupling reaction can employ a copper salt, such as copper(I) iodide, and also a bidentate phospine ligand, such as DPPE, in addition to the required transition metal catalyst, such as tris(dibenzylideneacetone)dipalladium(0). The groups A, B, X, and R¹-R¹² are as defined above unless otherwise noted below.

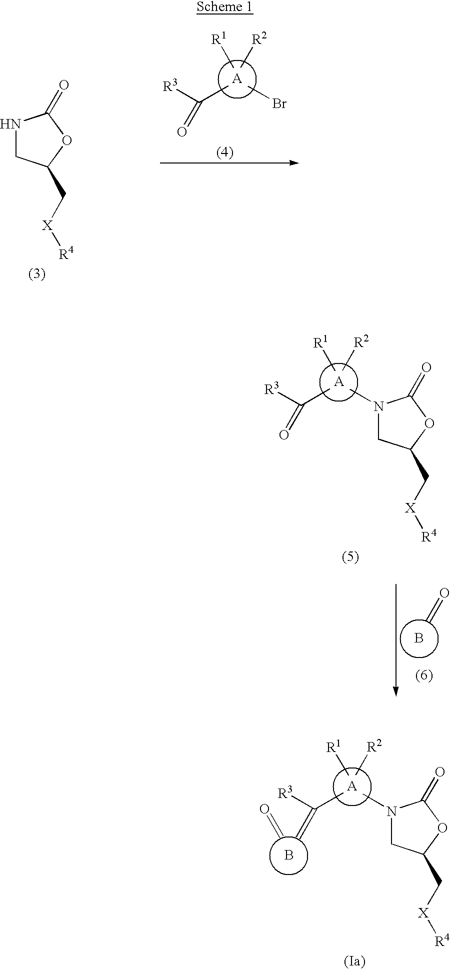

Scheme 1 shows the synthesis of compounds of formula (Ia) where R³ is hydrogen, alkoxy, alkyl, aryl, or haloalkoxy. Compounds of formula (3) where X is O and R⁴ is hydrogen can be converted to compounds of formula (3) where X is O, S, S(O), SO₂, or NR⁵ and R⁴ is alkanoyl, aryl, aryloyl, heteroaryl, and heteroaryloyl by methods known to those of ordinary skill in the art. These compounds can be reacted with compounds of formula (4) (where R³ is hydrogen, alkoxy, alkyl, aryl, haloalkoxy, or hydroxy) in the presence of a palladium catalyst and base to provide compounds of formula (5) where R³ is hydrogen, alkoxy, alkyl, aryl, hydroxy, or haloalkoxy. Representative palladium catalysts include Pd₂(dba)₃ with BINAP, (DPPF)PdCl₂, and (o-tolyl₃P)₂PdCl₂, while representative bases include Cs₂CO₃ and NaOt-Bu. Examples of solvents used in these reactions include toluene, THF, and dioxane. The reaction is conducted at about 80° C. to about 110° C. and reaction times are typically about 12 to about 36 hours.

Compounds of formula (5) (where R³ is hydrogen, alkyl, aryl, alkoxy, or hydroxy) can be condensed with compounds of formula (6) in the presence of base to provide compounds of formula (Ia) where R³ is hydrogen, alkyl, aryl, or hydroxy. Representative bases include piperidine, pyridine, and 2,6-lutidine. Examples of solvents used in these reactions include ethanol, isopropanol, and n-propanol. The reaction is conducted at about 90° C. to about 110° C. and reaction times are typically about 12 to about 48 hours.

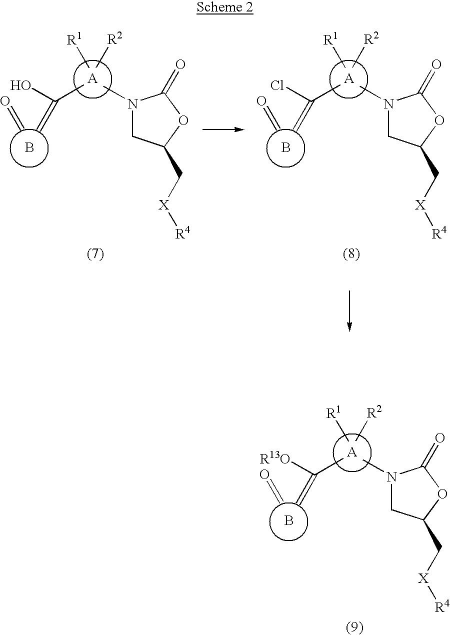

Scheme 2 shows the synthesis of compounds of formula (8). Compounds of formula (7) can be prepared from compounds of formula (5), wherein R³ is hydroxy or alkoxy, using methods known to those of ordinary skill in the art. Compounds of formula (7) can be reacted with a chlorinating reagent to provide compounds of formula (8). Representative chlorinating reagents include POCl₃ and PCl₅.

Compounds of formula (8) can be converted to compounds of formula (9) where R¹³ is alkyl or haloalkyl by treatment with an appropriately substituted alcohol in the presence of base. Representative bases include triethylamine and diisopropylethylamine.

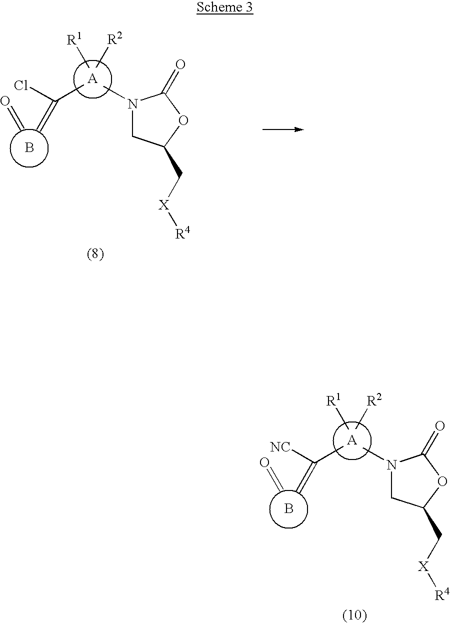

As shown in Scheme 3, compounds of formula (8) can be converted to compounds of formula (10) by a palladium-catalyzed coupling with Zn(CN)₂. Representative palladium catalysts include Pd(PPh₃)₄, Pd(PPh₃)₂Cl₂, and Pd₂(dba)₃ with TFP.

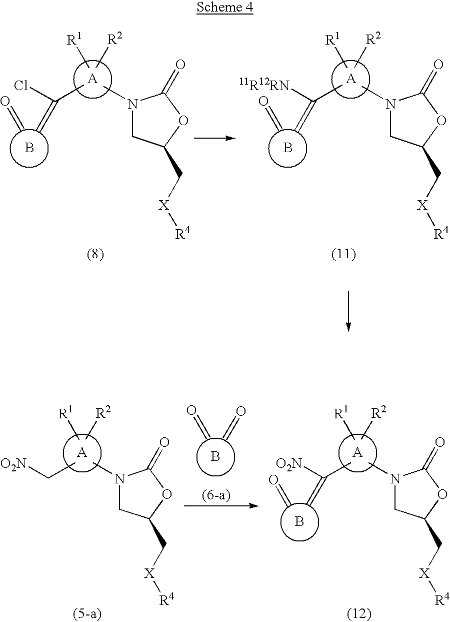

As shown in Scheme 4, compounds of formula (8) can be converted to compounds of formula (11) by reaction with an appropriately substituted amine (HNR¹¹R¹²) in the presence of base. Representative bases include triethylamine, diisopropylethylamine, and an excess of the amine reagent.

Compounds of formula (11) where R¹¹ and R¹² are hydrogen can be oxidized to compounds of formula (12) by a variety of methods known to those of ordinary skill in the art. Alternatively, compounds of formula (12) may be obtained by treatment of a nitromethylphenyl compound of formula (5-a) with a dicarbonyl compound of formula (6-a), for example isatin, and a base in an appropriate solvent.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects. It should be noted that where mixtures of E and Z isomers were present hereinbelow, the dominant isomer of the thermodynamic mixture was reported, with the understanding that the other isomer was present in minor amount. Where appropriate, the separated isomers were distinguished.

### EXAMPLE 1

### N-(((5S)-3-(3-fluoro-4-((E)-(2-oxo-1,2-dihydro-3H-pyrrolo(2,3-b)pyridin-3-ylidene)methyl)phenyl)-2-oxo- 1,3-oxazolidin-5-yl)methyl)acetamide

### EXAMPLE 1A

### ((5R)-2-oxo-1,3-oxazolidin-5-yl)methyl 2-nitrobenzenesulfonate

A solution of (5R)-5-(hydroxymethyl)-1,3-oxazolidin-2-one (1.2 g, prepared as described in Tetrahedron: Asymmetry 1995, 6, 1181-1190) in pyridine (5 mL) at −10° C. was treated with a solution of 2-nitrobenzensulfonyl chloride (2.75 g) in pyridine (3 mL), warmed to −5° C., stirred for 2.5 hours, treated with water, and extracted with ethyl acetate. The extract was washed with saturated sodium bicarbonate and brine, dried (MgSO₄), filtered, and concentrated. The concentrate was triturated with 1:3 acetone/hexanes to provide the desired product.

MS (APCI(+)) m/e 303 (M+H)⁺;

¹H NMR (300 MHz, DMSO-d₆) δ 8.15 (m,2H), 8.04 (t, 1H), 7.96 (t, 1H), 7.60 (br s, 1H), 4.83 (m, 1H), 4.48-4.34 (m, 2H), 3.57-3.51 (t, 1H), 3.22 (dd, 1H).

### EXAMPLE 1B

### N-(2,4-dimethoxyphenyl)-N-(((5R)-2-oxo-1,3-oxazolidin-5-yl)methyl)acetamide

A solution of Example 1A (12.0 g) in acetonitrile (50 mL) at room temperature was treated with 2,4-dimethoxybenzylamine (7.2 mL) and stirred for 72 hours. The reaction mixture was concentrated, dissolved in dichloromethane (20 mL), treated with pyridine (13.0 mL) and acetic anhydride (12.5 mL), stirred for 20 hours, and concentrated. The concentrate was purified by flash column chromatography on silica gel with 2:98 methanol/dichloromethane to 5:95 methanol/dichloromethane to provide the desired product.

MS (APCI(+)) m/e 309 (M+H)⁺;

¹H NMR (500 MHz, DMSO-d₆) (rotamers) δ 7.56 and 7.46 (br s, 1H), 6.95 (d, 1H), 6.60 and 6.54 (s, 1H), 6.53 and 6.47 (dd, 1H), 4.76 and 4.56 (m, 1H), 4.51 and 4.42 (m, 2H), 3.79 and 3.75 (s, 6H), 3.35 and 3.30 (m, 1H), 3.62 and 3.59 (m, 1H), 3.11 and 3.06 (m, 1H), 3.50 and 3.45 (m, 1H), 2.09 and 2.05 (s, 3H);

¹³C NMR (125 MHz, DMSO-d₆) (rotamers) δ 170.7 and 170.0, 160.1 and 159.6, 158.4 and 158.3, 157.9 and 157.8, 128.6 and 128.2, 117.3 and 116.9, 104.5, 98.6 and 98.2, 74.0 and 74.1, 55.3, 55.2, 51.1 and 47.8, 47.6 and 42.6, 42.8 and 42.6, 21.4 and 21.2.

### EXAMPLE 1C

### N-(2,4-dimethoxybenzyl)-N-(((5S)-3-(3-fluoro-4-formylphenyl)-2-oxo-1,3-oxazolidin-5-yl)methyl)acetamide

A suspension of BINAP (185 mg) and tris(dibenzylidenacetone)dipalladium (150 mg) in toluene (15 mL) in a sealable tube was degassed with nitrogen and treated sequentially with cesium carbonate (1.95 g), Example 1B (1.22 g), and 4-bromo-2-fluorobenzaldehyde (1.2 g). The tube was sealed and the mixture was heated to 90° C. for 24 hours, cooled, poured into 1:1 saturated ammonium chloride/water (100 mL), and extracted with ethyl acetate. The combined extracts were washed with brine and water, dried (MgSO₄), filtered, and concentrated. The concentrate was purified by flash column chromatography on silica gel with 8:1 hexanes/acetone to provide the desired product.

MS (ESI(+)) m/e 431 (M+H)⁺;

¹H NMR (300 MHz, CDCl₃) δ 10.27 (s, 1H), 7.88 (t, 1H), 7.62 (dd, 1H), 7.25 (dd, 1H), 6.98 (d, 1H), 6.45 (m, 2H), 4.85 (m, 1H), 4.60 (q, 2H), 4.06 (t, 1H), 3.86 (m, 2H), 3.80 (s, 6H), 3.50 (dd, 1H), 2.22 (s, 3H).

### EXAMPLES 1D and 1E

### N-(2,4-dimethoxybenzyl)-N-(((5S)-3-(3-fluoro-4-((E)-(2-oxo-1,2-dihydro-3H-pyrrolo(2,3-b)pyridin-3-ylidene)methyl)phenyl)-2-oxo-1,3 -oxazolidin-5-yl)methyl)acetamide and

### N-(2,4-dimethoxybenzyl)-N-(((5S)-3-(3-fluoro-4-((Z)-(2-oxo-1,2-dihydro-3H-pyrrolo(2,3-b)pyridin-3-ylidene)methyl)phenyl)-2-oxo-1,3-oxazolidin-5-yl)methyl)acetamide

A suspension of Example 1C (200 mg) in ethanol (3 mL) was treated with 1,3-dihydro-2H-pyrrolo(2,3-b)pyridin-2-one (81 mg), and piperidine (15 μL), sealed, heated to 100° C. for 24 hours, cooled to room temperature, and concentrated. . The concentrate was purified by flash column chromatography on silica gel with 2:98 methanol/dichloromethane to provide the desired product.

MS (ESI(+)) m/e 548 (M+H)⁺;

¹H NMR (300 MHz, DMSO-d₆) δ 8.10 (t, 1H), 7.82 (t, 1H), 7.62 (m, 2H), 7.50 (d, 1H), 7.02-6.89 (m, 2H), 6.62-6.45 (m, 4H), 4.88 (m, 1H), 4.54-4.42 (m, 2H), 4.16 (t, 1H), 3.80 (m, 2H), 3.75 (s, 6H), 3.52 (m, 1H), 2.05 (s, 3H).

### EXAMPLE 1F

### N-(((5S)-3-(3-fluoro-4-((E)-(2-oxo-1,2-dihydro-3H-pyrrolo(2,3-b)pyridin-3-ylidene)methyl)phenyl)-2-oxo-1,3-oxazolidin-5-yl)methyl)acetamide

A solution of Examples 1D and 1E (160 mg) in 1:1 dichloromethane/trifluoroacetic acid (6 mL) at room temperature was stirred for 2 hours and concentrated. The concentrate was purified by reverse phase HPLC (gradient 10%-95% (0.1% TFA/H₂O-acetonitrile) to provide the desired product as a 2:1 mixture of E and Z isomers.

MS (ESI(+)) m/e 397 (M+H)⁺;

¹H NMR (300 MHz, DMSO-d₆) δ 11.15 and 11.10 (s, 1H), 8.65 (t, 1H), 8.22 (t, 1H), 8.10 and 8.06 (d, 1H), 7.82 (m, 1H), 7.70-7.62 (m, 2H), 7.54 and 7.34 (dd, 1H), 7.01 (dd, 1H), 6.93 (dd, 1H), 4.78 (m, 1H), 4.20 (t, 1H), 3.82 (t, 1H), 3.50 (m, 2H), 1.81 and 1.83 (s, 3H).

### EXAMPLE 2

### N-(((5S)-3-(4-((Z)-(4,5-dimethyl-2-oxo-1,2-dihydro-3H-indol-3-ylidene)methyl)phenyl)-2-oxo-1,3-oxazolidin-5-yl)methyl)acetamide

A mixture of N-(((5S)-3-(4-formylphenyl)-2-oxo-1,3-oxazolidin-5-yl)methyl)acetamide prepared as described in J Med. Chem. 1990, 33, 2569-2578 (55 mg), 4,5-dimethyl-1,3-dihydro-2H-indol-2-one (36 mg), piperidine (10 μL), and ethanol (3 mL) in a sealed tube was heated to 90° C. for 48 hours, cooled to room temperature, and concentrated. The concentrate was purified by flash column chromatography on silica gel with 95:5 dichloromethane/methanol to provide the desired product as a 2:1 mixture of Z and E isomers.

MS (DCI/NH₃) m/e 406 (M+H)⁺;

¹H NMR (500 MHz, DMSO-d₆) δ 10.40 (s, 1H), 8.24 (m, 1H), 8.18 (d, 2H), 7.74 (s, 0.66H), 7.60 (m, 2H), 7.58 (m, 0.33H), 7.00 (d, 1H), 6.58 (m, 1H), 4.78 (m, 1H), 4.20 (t, 1H), 3.80 (t, 1H), 3.44 (m, 2H), 2.48 (s, 3H), 2.12 (s, 3H), 1.84 (s, 3H).

### EXAMPLE 3

### N-(((5S)-2-oxo-3-(4-((E)-(2-oxo-1,2-dihydro-3H-indol-3-ylidene)methyl)phenyl)-1,3-oxazolidin-5-yl)methyl)acetamide

The desired product was prepared as a 2:1 mixture of E and Z isomers by substituting 1,3-dihydro-2H-indol-2-one for 4,5-dimethyl-1,3-dihydro-2H-indol-2-one in Example 2.

MS (APCI/NH₃) m/e 378 (M+H)⁺;

¹H NMR (300 MHz, DMSO-d₆) δ 10.58 (s, 1H), 8.25 (t, 1H), 7.75 (dd, 2H), 7.65 (m, 2H), 7.59 (s, 1H), 7.25 (t, 1H), 6.89 (d, 1H), 4.76 (m, 1H), 4.20 (t, 1H), 3.82 (dd, 1H), 3.45 (t, ¹H), 1.85 (s, 3H).

### EXAMPLE 4

### N-(((5S)-3-(4-((E)-(5-chloro-2-oxo-1,2-dihydro-3H-indol-3-ylidene)methyl)phenyl)-2-oxo-1,3-oxazolidin-5-yl)methyl)acetamide

The desired product was prepared as a 2:1 mixture of E and Z isomers by substituting 5-chloro-1,3-dihydro-2H-indol-2-one for 4,5-dimethyl-1,3-dihydro-2H-indol-2-one in Example 2.

MS (APCI/NH₃) m/e 412 (M+H)⁺;

¹H NMR (300 MHz, DMSO-d₆) δ 10.72 (s, 1H), 8.25 (t, 1H), 7.75 (dd, 2H), 7.68 (s, 1H), 7.54 (d, 1H), 7.29 (dd, 1H), 6.90 (d, 1H), 4.77 (m, 1H), 4.21 (t, 1H), 3.84(dd, 1H), 3.46 (t, 1H), 1.85 (s, 3H).

### EXAMPLE 5

### N-(((5S)-3-(4-((E)-(5-nitro-2-oxo-1,2-dihydro-3H-indol-3-ylidene)methyl)phenyl)-2-oxo-1,3-oxazolidin-5-yl)methyl)acetamide

The desired product was prepared as a 2:1 mixture of E and Z isomers by substituting 5-nitro-1,3-dihydro-2H-indol-2-one for 4,5-dimethyl- 1,3-dihydro-2H-indol-2-one in Example 2.

MS (DCI/NH₃) m/e 423 (M+H)⁺; ¹H NMR (300 MHz, DMSO-d₆) δ 8.72 (d, 1H), 8.57 (t, 1H), 8.48 (d, 1H), 8.30-8.20 (m, 5H), 7.89-7.86 (m, 3H), 7.75 (dd, 3H), 7.69 (dd, 2H), 7.28 (m, 1H), 7.23 (m, 1H), 4,77 (m, 2H), 4.22 (m, 2H), 3.83 (m, 2H). 3.44 (m, 6H), 1.84-1.83 (m, 6H).

### EXAMPLE 6

### N-(((5S)-3-(3-fluoro-4-((E)-(2-oxo-1,2-dihydro-3H-indol-3-ylidene)methyl)phenyl)-2-oxo-1,3-oxazolidin-5-yl)methyl)acetamide

The desired product was prepared as a 2:1 mixture of E and Z isomers by substituting 1,3-dihydro-2H-indol-2-one for 1,3-dihydro-2H-pyrrolo(2,3-b)pyridin-2-one in Example 1.

MS (APCI/NH₃) m/e 396 (M+H)⁺;

¹H NMR (300 MHz, DMSO-d₆) δ 0.60 (s, 1H), 10.55 (s, 1H), 8.68 (t, 1H), 8.20 (t, 1H), 7.78 (t, 2H), 7.69 (s, 1H), 7.60 (m, 1H), 7.45 (s, 1H), 7.29 (m, 1H), 7.20 (m, 1H), 6.85 (t, 1H), 6.82 (d, 1H), 6.78 (m, 1H), 4.72 (m, 1H), 4.25 (t, 1H), 3.84 (dd, 1H), 3.46 (t, 1H), 1.85 (s, 3H).

### EXAMPLE 7

### N-(((5S)-3-(4-((E)-(3-methyl-2,5-dioxo-4-imidazolidinylidene)methyl)phenyl)-2-oxo-1,3-oxazolidin-5-yl)methyl)acetamide

The desired product was prepared by substituting 1-methyl-2,4-imidazolidinedione for 4,5-dimethyl-1,3-dihydro-2H-indol-2-one in Example 2.

MS (ESI(+)) m/e 359 (M+H)⁺;

¹H NMR (500 MHz, DMSO-d₆) δ 11.28 (s, 1H), 8.22 (s, 1H), 8.00 (d, 2H), 7.54 (d, 2H), 6.37 (s, 1H), 4.73 (m, 1H), 4.14 (t, 1H), 3.78 (m, 1H), 3.42 (m, 2H), 3.08 (s, 3H), 1.83 (s, 3H).

### EXAMPLE 8

### N-(((5S)-3-(4-((Z)-(2,4-dioxo-1,3-thiazolidin-5-ylidene)methyl)phenyl)-2-oxo-1,3-oxazolidin-5-yl)methyl)acetamide

A solution of N-(((5S)-3-(4-formylphenyl)-2-oxo-1,3-oxazolidin-5-yl)methyl)acetamide (0.03 g, prepared as described in J Med. Chem. 1990, 33, 2569-2578), piperidine (30 μL), and 1,3-thiazolidine-2,4-dione (0.03 g) in methanol (5 mL) was heated to 90 ° C. for 4 days, cooled to room temperature, and concentrated. The concentrate was triturated with ethanol, filtered, and dried under vacuum to provide the desired product.

MS (DCI/NH₃) m/e 362 (M+H)⁺;

¹H NMR (300 MHz, DMSO-d₆) δ 12.50 (br s,1H), 8.22 (t, 1H), 7.73 (s, 1H), 7.70 (d, 2H), 7.63 (d, 2H), 4.75 (m, 1H), 4.17 (t, 1H), 3.79 (dd, 1H), 3.43 (t, 2H), 1.83 (s, 3H).

### EXAMPLE 9

### N-(((5S)-3-(4-((Z)-(3-tert-butyl-1-methyl-5-oxo-1,5-dihydro-4H-pyrazol-4-ylidene)methyl)phenyl)-2-oxo-1,3-oxazolidin-5-yl)methyl)acetamide

The desired product was prepared by substituting 5-tert-butyl-2-methyl-2,4-dihydro-3H-pyrazol-3-one for 4,5-dimethyl-1,3-dihydro-2H-indol-2-one in Example 2.

MS (APCI(+)) m/e 399 (M+H)⁺;

¹H NMR (300 MHz, DMSO-d₆) δ 8.66 (d, 1H), 7.87 (s, 1H), 7.67 (dd, 1H), 7.28 (d, 1H), 6.99 (d, 1H), 5.56 (s, 1H), 4.77 (m, 1H), 4.21 (t, 1H), 4.01 (s, 2H), 3.83 (dd, 1H), 3.44 (m, 3H), 1.28 (s, 9H), 1.15(s, 3H).

### EXAMPLE 10

### N-(((5S)-3-(4-((Z)-(3-tert-butyl-5-oxo-1-phenyl-1,5-dihydro-4H-pyrazol-4-ylidene)methyl)phenyl)-2-oxo-1,3-oxazolidin-5-yl)methyl)acetamide

The desired product was prepared by substituting 5-tert-butyl-2-phenyl-2,4-dihydro-3H-pyrazol-3-one for 4,5-dimethyl-1,3-dihydro-2H-indol-2-one in Example 2. mp: 70° C.;

MS (ESI(+)) m/e 461 (M+H)⁺;

¹H NMR (300 MHz, DMSO-d₆) δ 8.66-8.64 (d, 1H), 8.22-8.18 (t, 1H), 7.93-7.90 (d, 1H), 8.83-7.80 (d, 1H), 7.70 (d, 2H), 7.46-7.40 (m, 4H), 7.20 (m, 2H), 7.09-7.06 (d, 1H), 5.75 (s, 1H), 4.70-4.65 (m, 1H), 4.09-4.03 (t, 1H), 3.87-3.79 (m, 1H), 1.82 (s, 3H), 1.43 (s, 9H).

### EXAMPLE 11

### N-(((5S)-3-(4-((Z)-(2,4-dioxo-1,3-thiazolidin-5-ylidene)methyl)-3-fluorophenyl)-2-oxo- 1,3-oxazolidin-5-yl)methyl)acetamide

The desired product was prepared by substituting 1,3-thiazolidine-2,4-dione for 1,3-dihydro-2H-pyrrolo(2,3-b)pyridin-2-one in Example 1.

MS (APCI(+)) m/e 380 (M+H)⁺;

¹H NMR (500 MHz, DMSO-d₆) δ 12.68 (s, 1H), 8.22 (t, 1H), 7.76 (s, 1H), 7.64 (d, 1H), 7.58 (m, 1H), 7.50 (d, 1H), 4.68(m, 1H), 4.16 (t, 1H), 3.78(m, 1H), 3.42 (m, 2H), 1.82 (s, 3H).

### EXAMPLE 12

### N-(((5S)-3-(3 -fluoro-4-((Z)-(1-methyl-2-oxo-1,2-dihydro-3H-indol-3-ylidene)methyl)phenyl)-2-oxo-1,3-oxazolidin-5-yl)methyl)acetamide

The desired product was prepared by as a 1:1 mixture of E and Z isomers by substituting 1-methyl-1,3-dihydro-2H-indol-2-one for 1,3-dihydro-2H-pyrrolo(2,3-b)pyridin-2-one in Example 1.

MS (ESI(+)) m/e 410 (M+H)⁺;

¹H NMR (300 MHz, DMSO-d₆) δ 8.25 (t, 1H), 7.83 (t, 1H), 7.70-7.66 (dd, 1H), 7.60 (s, 1H), 7.51-7.48 (dd,1 H), 7.38-7.36 (dd, 1H), 7.08 (s, 1H), 6.95 (t, 1H), 6.55 (s, 1H), 4.79 (m, 1H), 4.20 (t, 1H), 3.84-3.79 (m, 1H), 3.74 (s, 3H), 3.51 (s, 1H), 3.44 (t, 1H), 1.85 (s, 3H).

### EXAMPLE 13

### N-(((5S)-3-(3-fluoro-4-((E)-(1-methyl-5-nitro-2-oxo-1,2-dihydro-3H-indol-3-ylidene)methyl)phenyl)-2-oxo-1,3-oxazolidin-5-yl)methyl)acetamide

The desired product was prepared as a 5:1 mixture of E and Z isomers by substituting 1-methyl-5-nitro-1,3-dihydro-2H-indol-2-one for 1,3-dihydro-2H-pyrrolo(2,3-b)pyridin-2-one in Example 1.

MS (ESI(+)) m/e 455 (M+H)⁺;

¹H NMR (300 MHz, DMSO-d₆) δ 8.33-8.30 (dd,1H), 8.26 (t,1H), 8.21-8.20 (m,1H), 7.93 (t,1H), 7.81 (s,1H), 7.74-7.70 (dd,1H), 7.58-7.55 (dd,1H), 7.32-7.29 (d, 1H), 4.83-4.78 (m,1H), 4.22 (t, 1H), 3.84 (m, 1H), 3.74 (s, 1H), 3.51-3.44 (m, 3H), 1.84 (s, 3H).

### Example 14

### N-(((5S)-3-(4-((E)-(6-chloro-2-oxo-1,2-dihydro-3H-indol-3-ylidene)methyl)-3-fluorophenyl)-2-oxo-1,3-oxazolidin-5-yl)methyl)acetamide

The desired product was prepared by substituting 6-chloro-1,3-dihydro-2H-indol-2-one for 1,3-dihydro-2H-pyrrolo(2,3-b)pyridin-2-one in Example 1.

MS (ESI(+)) m/e 430 (M+H)⁺;

¹H NMR (300 MHz, DMSO-d₆) δ 10.80 (s, 1H), 8.25 (t, ¹H), 7.82 (t, 1H), 7.69-7.65 (dd, 1H), 7.55 (s, 1H), 7.51-7.48 (dd, 1H), 7.35-7.32 (d, 1H), 6.97-6.93 (dd, 1H), 6.90 (m, 1H), 4.97 (m, 1H), 4.20 (t, 1H), 3.84-3.79 (m, 1H), 3.74 (s, 1H), 3.51-3.42 (m, 2H), 1.85 (s, 3H).

### EXAMPLE 15

### N-(((5S)-3-(4-((E)-(5-methoxy-2-oxo-1,2-dihydro-3H-indol-3-ylidene)methyl)-3-fluorophenyl)-2-oxo-1,3-oxazolidin-5-yl)methyl)acetamide

The desired product was prepared by substituting 5-methoxy-1,3-dihydro-2H-indol-2-one for 1,3-dihydro-2H-pyrrolo(2,3-b)pyridin-2-one in Example 1.

MS (ESI(+)) m/e 426 (M+H)⁺;

¹H NMR (300 MHz, DMSO-d₆) δ 10.44 (s, 1H), 8.25 (t, 1H), 7.84 (t, 1H), 7.70-7.65 (dd, 1H), 7.50 (m, 1H), 6.92 (br s, 1H), 6.85 (m, 1H), 6.81 (s, 1H), 4.78 (m, 1H), 4.20 (t, 1H), 3.82 (m, 1H), 3.74 (s, 1H), 3.63 (s, 3H), 3.44 (t, 2H), 1.84 (s, 3H).

### EXAMPLE 16

### N-(((5S)-3-(4-((Z)-(5-chloro-2-oxo-1,2-dihydro-3H-indol-3-ylidene)methyl)-3-fluorophenyl)-2-oxo-1,3-oxazolidin-5-yl)methyl)acetamide

The desired product was prepared as a 7:1 mixture of Z and E isomers by substituting 5-chloro-1,3-dihydro-2H-indol-2-one for 1,3-dihydro-2H-pyrrolo(2,3-b)pyridin-2-one in Example 1.

MS (ESI(+)) m/e 430 (M+H)⁺;

¹H NMR (300 MHz, DMSO-d₆) δ 10.72 (s, 1H), 8.70 (t, 1H), 7.87 (s, 1H), 7.61 (d, 1H), 7.36-7.32 (dd, 1H), 7.27-7.24 (dd, 1H), 6.84 (d, 1H), 4.77 (m, 1H), 4.19 (t, 1H), 3.80 (t, 1H), 3.74 (s, 2H), 3.44 (t, 1H), 1.84 (s, 3H).

### EXAMPLE 17

### N-(((5S)-3-(4-((E)-(1-methyl-2-oxo-1,2-dihydro-3H-indol-3-ylidene)methyl)phenyl)-2-oxo-1,3-oxazolidin-5-yl)methyl)acetamide

The desired product was prepared as a 3:2 mixture of E and Z isomers by substituting 1-methyl-1,3-dihydro-2H-indol-2-one for 4,5-dimethyl-1,3-dihydro-2H-indol-2-one in Example 2.

mp: 193-195° C.;

MS (DCI/NH₃) m/e 392 (M+H)⁺;

¹H NMR (300 MHz, DMSO-d₆) δ 8.50 (d, 1H), 8.24 (t, 1H), 7.83 (s, 1H), 7.79 (d, 2H), 7.75 (d, 2H), 7.71 (d, 2H), 7.69 (d,2H), 7.66 (d, 2H), 7.34 (t, 2H), 7.29 (d, 1H), 7.06 (d, 1H), 7.00 (d, 1H), 6.95 (d, 1H), 4.77 (m, 2H), 4.20 (t, 2H), 3.83 (m, 2H), 3.44 (m, 2H), 3.22 (d, 6H), 1.84 (s, 6H).

### EXAMPLE 18

### N-[((5S)-3-{4-[(E)-(6-chloro-2-oxo-1,2-dihydro-3H-indol-3-ylidene)methyl]phenyl}-2-oxo-1,3-oxazolidin-5-yl)methyl]acetamide

The desired product was prepared as a 2:1 mixture of E and Z isomers by substituting 6-chloro-1,3-dihydro-indol-2-one for 4,5-dimethyl-1,3-dihydro-2H-indol-2-one in Example 2.

MS (ESI(+)) m/e 412 (M+H)⁺;

¹H NMR (300MHz, DMSO-d₆) δ 10.74 (bs,1H), 8.5-8.4 (bd,1H), 8.4-8.3 (t,1H), 7.83-7.6 (m, 4H), 7.1-6.68 (m, 2H), 4.76 (m, 1H), 4.20 (t,1H), 3.83 (m,1H), 3.5-3.4 (m,2H), 1.85 (s,3H).

### EXAMPLE 19

### N-[((5S)-3-{4-[(E)-(5,6-dimethoxy-2-oxo-1,2-dihydro-3H-indol-3-ylidene)methyl]-3-fluorophenyl }-2-oxo-1,3-oxazolidin-5-yl)methyl]acetamide

The desired product was prepared as a 2:1 mixture of E and Z isomers by substituting 5,6-dimethoxy-1,3-dihydro-indol-2-one for 1,3-dihydro-2H-pyrrolo(2,3-b)pyridin-2-one in Example 1.

MS (ESI(+)) m/e 456 (M+H)⁺;

¹H NMR (300MHz, DMSO-d₆) δ 10.30 (s,1H), 8.64 (t,1H), 8.23 (t,3H), 7.60 (dd,1H), 7.57 (s, 1H), 7.38 (s, 1H), 7.30 (m, ¹H), 6.46 (s, 1H), 4.77 (m, 1H), 4.18 (t,1H), 3.80 (m, 1H), 3.78 (s,2H), 3.77 (s, 3H), 3.75 (s, 1H), 3.44 (m, 2H), 1.84 (s,1H).

### EXAMPLE 20

### N-[((5S)-2-oxo-3-{4-[(Z)-(4-oxo-2-thioxo-1,3-thiazolidin-5-ylidene)methyl]phenyl}-1,3-oxazolidin-5-yl)methyl]acetamide

The desired product was prepared by substituting 2-thioxo-4-thiazolidinone for 4,5-dimethyl-1,3-dihydro-2H-indol-2-one in Example 2.

MS (ESI(+)) m/e 377 (M+H)⁺;

¹H NMR (300MHz, DMSO-d₆) δ 8.28-8.2 (t,1H), 7.7 (s, 1H), 7.65 (s, 1H), 7.6 (S, 1H), 4.7-4.6 (m, 1H), 4.18 (t, 1H), 3.72- 3.67 (m, 1H), 3.43 (t, 2H), 3.33-3.28 (s, 3H), 1.3 (s, 3H).

### EXAMPLE 21

### N-[((5S)-3-{4-[(Z)-(5-methoxy-2-oxo-1,2-dihydro-3H-indol-3-ylidene)methyl]phenyl }-2-oxo-1,3-oxazolidin-5-yl)methyl]acetamide

The desired product was prepared as a 2:1 mixture of Z and E isomers by substituting 5-methoxy-1,3-dihydro-indol-2-one for 4,5-dimethyl-1,3-dihydro-2H-indol-2-one in Example 2.

MS (ESI(+)) m/e 408 (M+H)⁺;

¹H NMR (300MHz, DMSO-d₆) δ 10.36 (s, 1H), 8.48 (d, 2H), 8.23 (t, 1H), 7.78 (s, 1H), 7.78 (d, 2H), 7.37 (d, 1H), 6.78 (d, 1H), 6.72 (s, 1H), 4.76 (m, 1H), 4.19-3.81(m, 2H), 3.77 (s, 3H), 3.44 (t, 2H), 1.84 (s, 3H).

### EXAMPLE 22

### N-[((5S)-3-{4-[(E)-(1-acetyl-2-oxo-1,2-dihydro-3H-indol-3-ylidene)methyl]-3-fluorophenyl }-2-oxo-1,3-oxazolidin-5-yl)methyl]acetamide

The desired product was prepared as a 2:1 mixture of E and Z isomers by substituting 1-acetyl-1,3-dihydro-indol-2-one for 1,3-dihydro-2H-pyrrolo(2,3-b)pyridin-2-one in Example 1.

MS (DCI/NH₃) m/e 438 (M+H)⁺;

¹H NMR (300MHz, DMSO-d₆) δ 8.26 (t, 1H), 8.25 (t, 1H), 7.85 (t, 1H), 7.70 (s, 1H), 7.69 (dd, 1H), 7.50 (m, 1H), 7.41 (t, 1H), 7.16 (t, 1H), 4.79 (m, 1H), 4.21 (t, 1H), 3.82 (dd, 1H), 3.45 (t, 2H), 2.66 (s, 3H), 1.85 (s, 3H).

### EXAMPLE 23

### N-[((5S)-3-{4-[(Z)-(4,7-dimethyl-2-oxo-1,2-dihydro-3H-indol-3-ylidene)methyl]-3-fluorophenyl }-2-oxo-1,3-oxazolidin-5-yl)methyl]acetamide

The desired product was prepared by substituting 4,7-dimethyl-1,3-dihydro-indol-2-one for 1,3-dihydro-2H-pyrrolo(2,3-b)pyridin-2-one in Example 1.

MS (APCI/NH₃) m/e 424 (M+H)⁺;

¹H NMR (300MHz, DMSO-d₆) δ 10.55 (s, 1H), 8.38 (t, 1H), 8.23 (t, 1H), 7.63 (s, 1H), 7.60 (dd, 1H), 7.30 (dd, 1H), 6.95 (d, 1H), 6.72 (d, 1H), 4.77 (m, 1H), 4.18 (t, 1H), 3.78 (dd, 1H), 3.44 (m, 2H), 2.54 (s, 3H), 2.17 (s, 3H), 1.84 (s, 3H).

### EXAMPLES 24A and 24B

### N-{[(5S)-3-(3-fluoro-4-{(E)-[2-oxo-5-(trifluoromethoxy)-1,2-dihydro-3H-indol-3-ylidene]methyl}phenyl)-2-oxo-1,3-oxazolidin-5-yl]methyl}acetamide and

### N-{[(5S)-3-(3-fluoro-4-{(Z)-[2-oxo-5-(trifluoromethoxy)-1,2-dihydro-3H-indol-3-ylidene]methyl}phenyl)-2-oxo-1,3-oxazolidin-5-yl]methyl}acetamide

These two examples were prepared in the same experiment by substituting 5-trifluoromethoxy-1,3-dihydro-indol-2-one for 1,3-dihydro-2H-pyrrolo(2,3-b)pyridin-2- one in Example 1 and separating the two isomers by reverse phase HPLC (gradient 0%-95% (0.1% TFA/H₂O-acetonitrile).

Data for E isomer:

MS (DCI/NH₃) m/e 497 (M+NH₄)⁺;

¹H NMR (300MHz, DMSO-d₆) δ 6 10.85 (s, 1H), 8.25 (t, 1H), 8.25 (t, 1H), 7.83 (t, 1H), 7.68 (dd, 1H), 7.61 (s, 1H), 7.50 (dd, 1H), 7.28 (d, 1H), 7.21 (s, 1H), 6.97 (d, 1H), 4.78 (m, 1H), 4.20 (t, 1H), 3.82 (dd, 1H), 3.45 (t, 2H), 1.84 (s, 3H).

Data for Z isomer:

MS (DCI/NH₃) m/e 497 (M+NH₄)⁺;

¹H NMR (300 MHz, DMSO-d₆) δ 10.78 (s, 1H), 8.70 (t, 1H), 8.24 (t, 1H), 7.92 (s, 1H), 7.86 (s, 1H), 7.63 (dd, 1H), 7.36 (dd, 1H), 7.21 (d, 1H), 6.90 (s, 1H), 4.78 (m, 1H), 4.19 (t, 1H), 3.80 (dd, 1H), 3.44 (t, 2H), 1.84 (s, 3H).

It will be evident to one skilled in the art that the present invention is not limited to the forgoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims and therefore intended to be embraced therein.

What is claimed is

1. A compound of formula (I) 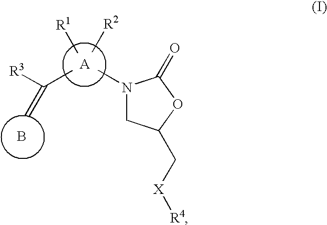
or a therapeutically acceptable salt thereof, wherein
A is selected from phenyl and a five- or six-membered aromatic ring containing one to three atoms selected from N, O, and S, and the remaining atoms are carbon, wherein A is substituted through carbon atoms in the ring;
B is heterocycle;
X is selected from the group consisting of O, S, S(O), SO₂, and NR⁵;
R¹ and R² are independently selected from the group consisting of hydrogen, alkoxy, alkyl, amino, cycloalkyl, halo, haloalkyl, hydroxy, and perfluoroalkyl;
R³ is selected from the group consisting of hydrogen, alkoxy, alkyl, amino, aryl, cyano, halo, haloalkoxy, hydroxy, and nitro;
R⁴ is selected from the group consisting of alkanoyl, alkoxycarbonyl, amido, aryl, aryloyl, heteroaryl, and heteroaryloyl; and
R⁵ is selected from the group consisting of hydrogen, alkyl, and arylalkyl;
with the proviso that when b is 2,4-dioxo-1,3-thiazolidin-5-yl and x is O, R⁴ is other than phenyl:

2. A compound according to claim 1 of formula (II) 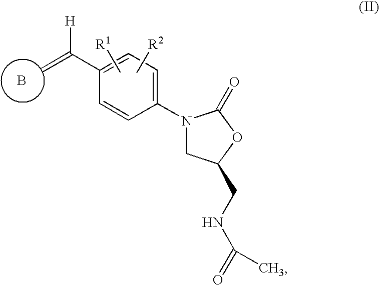
or a therapeutically acceptable salt thereof, wherein
B is heterocycle wherein the heterocycle is selected from the group consisting of 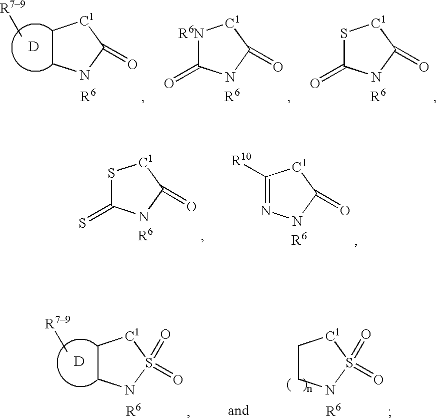
wherein C¹ is the point of attachment to the parent molecular moiety;
n is 1 or 2;
D is selected from phenyl and a five- or six-membered aromatic ring containing one or two atoms selected from N, O, and S, and the remaining atoms are carbon, wherein the N is optionally oxidized, and wherein D is fused through carbon atoms in the ring;
R¹ and R² are independently selected from the group consisting of alkoxy, alkyl, and halo;
each R⁶ is independently selected from the group consisting of hydrogen, alkanoyl, aikoxycarbonyl, alkyl, amido, aminoalkyl, aminosulfonyl, aryl, heteroaryl, hydroxyalkyl, and a nitrogen protecting group; and
R⁷-R¹⁰ are independently selected from the group consisting of hydrogen, alkoxy, alkoxycarbonyl, alkyl, amido, amino, aminosulfonyl, azido, carboxy, cyano, halo, haloalkoxy, haloalkyl, mercapto, nitro, perfluoroalkoxy, perfluoroalkyl, and thioalkoxy.

3. A compound according to claim 1 wherein A is phenyl.

4. A compound according to claim 3 wherein B is

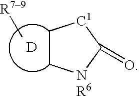

5. A compound according to claim 4 selected from the group consisting of
N-(((5S)-3-(3-fluoro-4-((E)-(2-oxo-1,2-dihydro-3H-pyrrolo(2,3-b)pyridin-3-ylidene)methyl)phenyl)-2-oxo-1,3-oxazolidin-5-yl)methyl)acetamide,
N-(((5S)-3-(4-((Z)-(4,5-dimethyl-2-oxo-1,2-dihydro-3H-indol-3-ylidene)methyl)phenyl)-2-oxo-1,3-oxazolidin-5-yl)methyl)acetamide,
N-(((5S)-2-oxo-3-(4-((E)-(2-oxo-1,2-dihydro-3H-indol-3-ylidene)methyl)phenyl)-1,3-oxazolidin-5-yl)methyl)acetamide,
N-(((5S)-3-(4-((Z)-(5-chloro-2-oxo-1,2-dihydro-3H-indol-3-ylidene)methyl)phenyl)-2-oxo-1,3-oxazolidin-5-yl)methyl)acetamide,
N-(((5S)-3-(4-((E)-(5-chloro-2-oxo-1,2-dihydro-3H-indol-3-ylidene)methyl)phenyl)-2-oxo- 1,3-oxazolidin-5-yl)methyl)acetamide,
N-(((5S)-3-(4-((E)-(5-nitro-2-oxo-1,2-dihydro-3H-indol-3-ylidene)methyl)phenyl)-2-oxo-1,3-oxazolidin-5-yl)methyl)acetamide,
N-(((5S)-3-(3-fluoro-4-((E)-(2-oxo-1,2-dihydro-3H-indol-3-ylidene)methyl)phenyl)-2-oxo-1,3-oxazolidin-5-yl)methyl)acetamide,
N-(((5S)-3-(3-fluoro-4-((Z)-(1-methyl-2-oxo-1,2-dihydro-3H-indol-3-ylidene)methyl)phenyl)-2-oxo-1,3-oxazolidin-5-yl)methyl)acetamide,
N-(((5S)-3-(3-fluoro-4-((E)-(1-methyl-5-nitro-2-oxo-1,2-dihydro-3H-indol-3 -ylidene)methyl)phenyl)-2-oxo-1,3-oxazolidin-5-yl)methyl)acetamide,
N-(((5S)-3-(4-((E)-(6-chloro-2-oxo-1,2-dihydro-3H-indol-3-ylidene)methyl)-3-fluorophenyl)-2-oxo-1,3-oxazolidin-5-yl)methyl)acetamide,
N-(((5S)-3-(4-((E)-(5-methoxy-2-oxo-1,2-dihydro-3H-indol-3-ylidene)methyl)-3-fluorophenyl)-2-oxo-1,3-oxazolidin-5-yl)methyl)acetamide,
N-(((5S)-3-(4-((Z)-(5-chloro-2-oxo-1,2-dihydro-3 H-indol-3-ylidene)methyl)-3-fluorophenyl)-2-oxo-1,3-oxazolidin-5-yl)methyl)acetamide,
N-(((5S)-3-(4-((E)-(1-methyl-2-oxo-1,2-dihydro-3H-indol-3-ylidene)methyl)phenyl)-2-oxo-1,3-oxazolidin-5-yl)methyl)acetamide,
N-[((5S)-3-{4-[(E)-(6-chloro-2-oxo-1,2-dihydro-3H-indol-3-ylidene)methyl]phenyl}-2-oxo-1,3-oxazolidin-5-yl)methyl]acetamide,
N-[((5S)-3-{4-[(E)-(5,6-dimethoxy-2-oxo-1,2-dihydro-3H-indol-3-ylidene)methyl]-3-fluorophenyl}-2-oxo- 1,3-oxazolidin-5-yl)methyl]acetamide,
N-[((5S)-3-{4-[(Z)-(5-methoxy-2-oxo-1,2-dihydro-3H-indol-3-ylidene)methyl]phenyl}-2-oxo-1,3-oxazolidin-5-yl)methyl]acetamide,
N-[((5S)-3-{4-[(E)-(1-acetyl-2-oxo-1,2-dihydro-3H-indol-3-ylidene)methyl]-3-fluorophenyl}-2-oxo-1,3-oxazolidin-5-yl)methyl]acetamide,
N-[((5S)-3-{4-[(Z)-(4,7-dimethyl-2-oxo-1,2-dihydro-3H-indol-3-ylidene)methyl]-3-fluorophenyl}-2-oxo-1,3-oxazolidin-5-yl)methyl]acetamide,
N-{[(5S)-3-(3-fluoro-4-{(E)-[2-oxo-5-(trifluoromethoxy)-1,2-dihydro-3H-indol-3-ylidene]methyl}phenyl)-2-oxo-1,3-oxazolidin-5-yl]methyl}acetamide, and
N-{[(5S)-3-(3-fluoro-4-{(Z)-[2-oxo-5-(trifluoromethoxy)-1,2-dihydro-3H-indol-3-ylidene]methyl}phenyl)-2-oxo-1,3-oxazolidin-5-yl]methyl}acetamide.

6. A compound according to claim 3 wherein B is

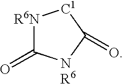

7. A compound according to claim 6 which is
N-(((5S)-3-(4-((E)-(3-methyl-2,5-dioxo-4-imidazolidinylidene)methyl)phenyl)-2-oxo-1,3-oxazolidin-5-yl)methyl)acetamide.

8. A compound according to claim 3 wherein B is

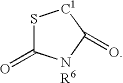

9. A compound according to claim 8 selected from the group consisting of
N-(((5S)-3-(4-((Z)-(2,4-dioxo-1,3-thiazolidin-5-ylidene)methyl)phenyl)-2-oxo-1,3-oxazolidin-5-yl)methyl)acetamide, and
N-(((5S)-3-(4-((Z)-(2,4-dioxo-1,3-thiazolidin-5-ylidene)methyl)-3-fluorophenyl)-2-oxo-1,3-oxazolidin-5-yl)methyl)acetamide.

10. A compound according to claim 3 wherein B is

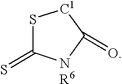

11. A compound according to claim 10 which is
N-[((5S)-2-oxo-3-{4-[(Z)-(4-oxo-2-thioxo-1,3-thiazolidin-5-ylidene)methyl]phenyl}-1,3-oxazolidin-5-yl)methyl]acetamide.

12. A compound according to claim 3 wherein B is

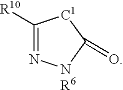

13. A compound according to claim 12 selected from the group consisting of
N-(((5S)-3-(4-((Z)-(3-tert-butyl-1-methyl-5-oxo-1,5-dihydro-4H-pyrazol-4-ylidene)methyl)phenyl)-2-oxo-1,3-oxazolidin-5-yl)methyl)acetamide, and
N-(((5S)-3-(4-((Z)-(3-tert-butyl-5-oxo-1-phenyl- 1,5-dihydro-4H-pyrazol-4-5ylidene)methyl)phenyl)-2-oxo-1,3-oxazolidin-5-yl)methyl)acetamide.

14. A pharmaceutical composition comprising a compound of claim 1 or a therapeutically acceptable salt thereof, in combination with a therapeutically acceptable carrier.

15. A method for treating bacterial infections in a patient in recognized need of such treatment comprising administering to the patient a therapeutically acceptable amount of a compound of claim 1, or a therapeutically acceptable salt thereof.

16. A method for treating psoriasis in a patient comprising administering to the patient a therapeutically acceptable amount of a compound of claim 1, or a therapeutically acceptable salt thereof.

17. A method for treating arthritis in a patient comprising administering to the patient a therapeutically acceptable amount of a compound of claim 1, or a therapeutically acceptable salt thereof.

18. A method for treating toxicity due to chemotherapy in a patient comprising administering to the patient a therapeutically acceptable amount of a compound of claim 1, or a therapeutically acceptable salt thereof.

19. A composition comprising a compound of claim 2, or a therapeutically acceptable salt thereof, and a therapeutically acceptable carrier.

20. A method for treating bacterial infections in a patient in recognized need of such treatment comprising administering to the patient a therapeutically acceptable amount of a compound of claim 2, or a therapeutically acceptable salt thereof.

21. A method for treating psoriasis in a patient comprising administering to the patient a therapeutically acceptable amount of a compound of claim 2, or a therapeutically acceptable salt thereof.

22. A method for treating arthritis in a patient comprising administering to the patient a therapeutically acceptable amount of a compound of claim 2, or a therapeutically acceptable salt thereof.

23. A method for treating toxicity due to chemotherapy in a patient comprising administering to the patient a therapeutically acceptable amount of a compound of claim 2, or a therapeutically acceptable salt thereof.

24. A compound selected from the group consisting of
N-(((5S)-3-(3-fluoro-4-((E)-(2-oxo-1,2-dihydro-3H-pyrrolo(2,3-b)pyridin-3-ylidene)methyl)phenyl)-2-oxo-1,3-oxazolidin-5-yl)methyl)acetamide,
N-(((5S)-3-(4-((Z)-(4,5-dimethyl-2-oxo-1 ,2-dihydro-3H-indol-3-5 ylidene)methyl)phenyl)-2-oxo- 1 ,3-oxazolidin-5-yl)methyl)acetamide,
N-(((5S)-2-oxo-3-(4-((E)-(2-oxo-1,2-dihydro-3H-indol-3-ylidene)methyl)phenyl)-1,3-oxazolidin-5-yl)methyl)acetamide,
N-(((5S)-3-(4-((Z)-(5-chloro-2-oxo-1,2-dihydro-3H-indol-3-ylidene)methyl)phenyl)-2-oxo-1,3-oxazolidin-5-yl)methyl)acetamide,
N-(((5S)-3-(4-((E)-(5-chloro-2-oxo-1,2-dihydro-3H-indol-3-ylidene)methyl)phenyl)-2-oxo- 1,3-oxazolidin-5-yl)methyl)acetamide,
N-(((5S)-3-(4-((E)-(5-nitro-2-oxo-1,2-dihydro-3H-indol-3-ylidene)methyl)phenyl)-2-oxo-1,3-oxazolidin-5-yl)methyl)acetamide,
N-(((5S)-3-(3-fluoro-4-((E)-(2-oxo-1,2-dihydro-3H-indol-3-ylidene)methyl)phenyl)-2-oxo-1,3-oxazolidin-5-yl)methyl)acetamide,
N-(((5S)-3-(4-((E)-(3-methyl-2,5-dioxo-4-imidazolidinylidene)methyl)phenyl)-2-oxo-1,3-oxazolidin-5-yl)methyl)acetamide,
N-(((5S)-3-(4-((Z)-(2,4-dioxo-1,3-thiazolidin-5-ylidene)methyl)phenyl)-2-oxo-1,3-oxazolidin-5-yl)methyl)acetamide,
N-(((5S)-3-(4-((Z)-(3-tert-butyl-1-methyl-5-oxo-1,5-dihydro-4H-pyrazol-4-ylidene)methyl)phenyl)-2-oxo-1,3-oxazolidin-5-yl)methyl)acetamide,
N-(((5S)-3-(4-((Z)-(3-tert-butyl-5-oxo-1-phenyl-1,5-dihydro-4H-pyrazol-4-ylidene)methyl)phenyl)-2-oxo-1,3-oxazolidin-5-yl)methyl)acetamide,
N-(((5S)-3-(4-((Z)-(2,4-dioxo-1,3-thiazolidin-5-ylidene)methyl)-3-fluorophenyl)-2-oxo-1,3-oxazolidin-5-yl)methyl)acetamide,
N-(((5S)-3-(3-fluoro-4-((Z)-(1-methyl-2-oxo-1,2-dihydro-3H-indol-3-ylidene)methyl)phenyl)-2-oxo-1,3-oxazolidin-5-yl)methyl)acetamide,
N-(((5S)-3-(3-fluoro-4-((E)-(1-methyl-5-nitro-2-oxo-1,2-dihydro-3H-indol-3-ylidene)methyl)phenyl)-2-oxo-1,3-oxazolidin-5-yl)methyl)acetamide,
N-(((5S)-3-(4-((E)-(6-chloro-2-oxo-1,2-dihydro-3H-indol-3-ylidene)methyl)-3-fluorophenyl)-2-oxo- 1,3-oxazolidin-5-yl)methyl)acetamide,
N-(((5S)-3-(4-((E)-(5-methoxy-2-oxo-1,2-dihydro-3H-indol-3-ylidene)methyl)-3-fluorophenyl)-2-oxo-1,3-oxazolidin-5-yl)methyl)acetarnide,
N-(((5S)-3-(4-((Z)-(5-chloro-2-oxo-1,2-dihydro-3H-indol-3-ylidene)methyl)-3-fluorophenyl)-2-oxo-1,3-oxazolidin-5-yl)methyl)acetamide,
N-(((5S)-3-(4-((E)-(1-methyl-2-oxo-1,2-dihydro-3H-indol-3-ylidene)methyl)phenyl)-2-oxo-1 ,3-oxazolidin-5-yl)methyl)acetamide,
N-[((5S)-3-{4-[(E)-(6-chloro-2-oxo-1,2-dihydro-3H-indol-3-ylidene)methyl]phenyl}-2-oxo-1,3-oxazolidin-5-yl)methyl]acetamide,
N-[((5S)-3-{4-[(E)-(5,6-dimethoxy-2-oxo-1,2-dihydro-3H-indol-3-ylidene)methyl]-3-fluorophenyl}-2-oxo- 1,3-oxazolidin-5-yl)methyl]acetamide,
N-[((5S)-2-oxo-3-{4-[(Z)-(4-oxo-2-thioxo- 1,3-thiazolidin-5-ylidene)methyl]phenyl}-1,3-oxazolidin-5-yl)methyl]acetamide,
N-[((5S)-3-{4-[(Z)-(5-methoxy-2-oxo-1,2-dihydro-3H-indol-3-ylidene)methyl]phenyl}-2-oxo-1,3-oxazolidin-5-yl)methyl]acetamide,
N-[((5S)-3-{4-[(E)-(1-acetyl-2-oxo-1,2-dihydro-3H-indol-3-ylidene)methyl]-3-fluorophenyl}-2-oxo-1,3-oxazolidin-5-yl)methyl]acetamide,
N-[((5S)-3-{4-[(Z)-(4,7-dimethyl-2-oxo-1,2-dihydro-3H-indol-3-ylidene)methyl]-3-fluorophenyl}-2-oxo-1,3-oxazolidin-5-yl)methyl]acetamide,
N-{[(5S)-3-(3-fluoro-4-{(E)-[2-oxo-5-(trifluoromethoxy)- 1,2-dihydro-3H-indol-3-ylidene]methyl}phenyl)-2-oxo-1,3-oxazolidin-5-yl]methyl}acetamide, and
N-{[(5S)-3-(3-fluoro-4-{(Z)-[2-oxo-5-(trifluoromethoxy)- 1,2-dihydro-3H-indol-3-ylidene]methyl}phenyl)-2-oxo- 1,3-oxazolidin-5-yl]methyl}acetamide.
